(19) United States
(12) Patent Application Publication
(10) Pub. No.: US 2013/0237440 A1
(43) Pub. Date: Sep. 12, 2013

(54) METHOD AND MEANS FOR IDENTIFICATION OF ANIMAL SPECIES

(71) Applicant: CITY UNIVERSITY OF HONG KONG, Hong Kong (CN)

(72) Inventors: Shuk Han CHENG, Honk Kong (CN); Chun Chi Lin, Hong Kong (CN)

(73) Assignee: CITY UNIVERSITY OF HONG KONG, Hong Kong (CN)

(21) Appl. No.: 13/777,308

(22) Filed: Feb. 26, 2013

Related U.S. Application Data
(60) Provisional application No. 61/608,824, filed on Mar. 9, 2012.

(51) Int. Cl. C12Q 1/68

(52) U.S. Cl. CPC C12Q 1/6888
USPC 506/9; 506/16

(57) ABSTRACT

There is provided a method of rapid identification of a mammalian species origin or mammalian species origins of a sample. The method includes but not limited to the steps of 1) gathering a sample to be tested, 2) processing the sample for use for identification purpose, 3) dividing the sample into a number of portions for situation in a multi-well container or containers, 4) providing a plurality of DNA probes, at least some of the DNA probes having DNA sequences with SEQ ID NOs. 1-241, wherein the number of the sample portions is greater than the number of mammalian species types from which the DNA probes derives.

### RELATED APPLICATION

This application claims priority from U.S. Patent Application Ser. No. 61/608,824 filed on Mar. 9, 2012, contents of which are incorporated in this patent application in its entirety.

### FIELD OF THE INVENTION

The present invention is concerned with a method of identification of species, and in particular a method of making relatively rapid identification of animal species including but not limited to Bos Taurus (cattle), Sus scrofa (pig), Ovis aries (sheep), Equus caballus (horse), Canis lupus (dog) and Mus musculus (mouse) and Felis Catus (cat). The present invention is also concerned with means for use in carrying out such method, and a method for engineering such means.

### BACKGROUND OF THE PRESENT INVENTION

Due to a vast range of reasons, there has been a need of identifying animal species origin of a sample. For example, in the context of forensic investigation there is often the need of ascertaining the identity of an animal tissue sample in a crime scene. In the context of quality control of food manufacturing, there is a need to ascertain whether a food product claimed to contain certain type of animal meat is indeed what it claims to be. There are actually many other possible applications of using animal species origin or animal tissue identification methods, and one can also envisage that such applications can be very useful in medical science and pharmaceutical industry, and of course to address food safety issues as well.

There have been many methodologies seeking to address the needs of identification of animal species. While many of these methodologies have their unique characteristics, they often suffer from drawbacks, and tend to address some issues but not others. For example, some methodologies tend to be fairly accurate in their identification but at the expense of tremendous complexity in procedures. Some other methodologies are relatively simple in terms of procedures but often involve using high end equipment which requires significant financial investment and specially trained personnel (e.g. high speed DNA sequencing). Yet some methodologies are easy to grasp but the time needed to conduct identification is too long and commercially unrealistic and thus virtually unusable. Yet further, some methodologies are not able to distinguish a specimen when the sample contains multiple sources of animal species tissues, or at least they are not able to distinguish a specimen in one experiment.

The present invention seeks to address the aforementioned issues by providing a method which endeavors to balance different factors, or at least to provide an alternative balance, and means thereof. At least the present invention seeks to provide an alternative to the public for identifying an animal species sample, and means thereof.

### SUMMARY OF THE INVENTION

According to a first aspect of the present invention, there is provided a method of rapid identification of a mammalian species origin or mammalian species origins of a sample, comprising the steps of:
a) gathering a sample to be tested;
b) processing the sample for use for identification purpose;
c) dividing the sample into a number of portions for situation in a multi-well container or containers;
d) providing a plurality of DNA probes, at least some of the DNA probes having DNA sequences with SEQ ID NOs. 1-241 as shown in below Appendix and the accompanied Sequence Listing or at least 98% sequence identify of the DNA sequences thereof, wherein the number of the sample portions is greater than the number of mammalian species types from which the DNA probes derives;
e) selecting some or all of the DNA probes for hybridization with the sample portions;
f) subjecting the sample portions for intended hybridization with the selected DNA probes simultaneously;
g) detecting for positive hybridization results from the intended hybridization of the sample portions and the selected DNA probes; and
h) assessing the mammalian species origin or origins of the sample according to positive hybridization results.

According to a second aspect of the present invention, there is provided a kit for use in rapid identification of mammalian species origin or mammalian species origins of a sample, comprising a plurality of DNA probes derived from a number of different mammalian species, at least some of the DNA probes having DNA sequences with SEQ ID NOs. 1-241. In an embodiment, the DNA probes may be immobilized on a suitable substrate, such as on a hybridization membrane or on a wall of a plastic(s) well.

According to a third aspect of the present invention, there is provided a method of engineering DNA probes for use in rapid identification of mammalian species origin or mammalian species origins of a sample, comprising the steps of:
a) identifying regions of DNA sequence with a length from 60 to 80 bases from 48^{th} to 705^{th} bp of the COI gene of a first mammalian species, yielding a first group of DNA regions; and
b) from the first group of DNA regions, identifying regions of DNA sequence meeting the following criteria:
   i) with GC content from substantially 50 to 52%;
   ii) with a positive value of delta G, yielding a third group of DNA regions;
   iii) in which the hybridization temperature (Thyb) is from 15-25° C. below the melting temperature (Tm); and
   iv) in which the difference between the number of secondary structures (SS) and the value of secondary structure (SS) is between 0 to 4; and
c) engineering DNA probes based on the identified DNA regions from said step b).

Preferably, the delta G value may be larger than 1. The Tm may be substantially 79-83° C. With 50 mmole of sodium chloride and without any organic solvent, the Thyb may be larger than 60° C., NaCl w/o any organic solvent. In a specific embodiment, at least some of the DNA probes may have DNA sequences with SEQ ID NOs. 1-241.

According to a fourth aspect of the present invention, there is provided a method of engineering DNA probes for use in rapid identification of mammalian species origin or mammalian species origins of a sample, comprising the steps of:
a) identifying regions of DNA sequence with a length from 60 to 80 bases from 48^{th} to 705^{th} bp of the COI gene of a first mammalian species, yielding a first group of DNA regions;
b) from the first group of DNA regions, selecting regions of DNA sequence with GC content from substantially 50 to 52%, yielding a second group of DNA regions;
c) from the second group of DNA regions, selecting regions of DNA sequence with a positive value of delta G, yielding a third group of DNA regions;
d) from the third group of DNA regions, selecting regions of DNA sequence in which the hybridization temperature (Thyb) is from 15-25° C. below the melting temperature (Tm), yielding a fourth group of DNA regions;
e) from the fourth group of DNA regions, selecting regions of DNA sequence in which the difference between the number of secondary structures (SS) and the value of secondary structure (SS) is between 0 to 4; and
f) engineering DNA probes based on the identified DNA regions from said step e).

Preferably, the delta G value may be larger than 1. The Tm may be substantially 79-83° C. With 50 mmole of sodium chloride and without any organic solvent, the Thyb may be larger than 60° C., NaCl w/o any organic solvent. In a specific embodiment, at least some of the DNA probes may have DNA sequences with SEQ ID NOs. 1-241.

### BRIEF DESCRIPTION OF DRAWINGS

Some embodiments of the present invention will now be explained, with reference to the accompanied drawings, in which:

FIG. 1 shows images illustrating specificity examination of species-specific probes of each listed mammal species according to an embodiment of the present invention;

FIG. 2 is a key showing the pattern of distribution of species specific probes immobilized on the membranes used in experiments, results of which are demonstrated in FIG. 1 and FIGS. 3a to 3d;

FIGS. 3a to 3d show the effect of hybridization temperature and/or time for hybridization according to an embodiment of the present invention;

FIG. 4 is a key showing the pattern of distribution of species specific probes immobilized on the membrane for multiple species detection in an experiment, results of which are demonstrated in FIG. 5; and

FIG. 5 shows the effect of multiple species detection according to an embodiment of the present invention.

### DETAILED DESCRIPTION OF PREFERRED EMBODIMENTS OF THE INVENTION

DNA probes or DNA oligo-nucleotides (or oligo-nucleotide sequences) have been widely used for a vast range of applications. The requirements for a DNA probe or a group of DNA probes suitable for use in different applications can be rather different. The usefulness, accuracy and/or efficiency of the applications largely depend on the DNA probe sequence(s). While there has been numerous proposal of making or using DNA probes in different scenario, there is however, no one single principle that can be appropriate for use in all applications. In this context, after much research and development, a number of DNA probes have been engineered, allowing identification of mammalian species including Bos Taurus (cattle), Sus scrofa (pig), Ovis aries (sheep), Equus caballus (horse), Canis lupus (dog) and Mus musculus (mouse) and Felis Catus (cat). Accordingly, in an embodiment of the present invention, there is provided a plurality of DNA probes having DNA sequences with SEQ ID NOs. 1-241, as shown in Appendix and the Sequence Listing submitted with this patent specification. It is to be noted that the Appendix shows sequences generally mirroring those in the Sequence Listing although in the Appendix further designations are provided for reference and illustration purposes.) Studies have shown that these particular sequences are sufficiently species specific and are able to detect the relevant species origin of a sample or multiple species origins of a sample, as the case may be. Studies have also shown that sequences having at least 98% sequence identify will likewise produce workable results.

One object of this present invention is to allow rapid identification of a mammalian species origin or a mammalian species origin in one experiment or simultaneously, and efficiently, without having to make use of costly equipment. Accordingly, in one embodiment of the present invention there is provided a method of rapid identification of a mammalian species origin or mammalian species origins of a sample, comprising the steps of a) gathering a sample to be tested, b) processing the sample for use for identification purpose, c) dividing the sample into a number of portions for situation in a multi-well container or containers; d) providing a plurality of DNA probes, at least some of the DNA probes having DNA sequences with SEQ ID NOs. 1-241, wherein the number of the sample portions is greater than the number of mammalian species types from which the DNA probes derives; e) selecting some or all of the DNA probes for hybridization with the sample portions; f) subjecting the sample portions for intended hybridization with the selected DNA probes simultaneously; g) detecting for positive hybridization results from the intended hybridization of the sample portions and the selected DNA probes; and i) assessing the mammalian species origin or origins of the sample according to positive hybridization results.

It is to be noted that with this embodiment, a skilled person would be able to conduct one experiment for detecting the species origin of a sample. Specifically, referring to step f) of subjecting the sample portions for intended hybridization with the selected DNA probes simultaneously, multiple hybridizations can occur simultaneously in one experiment. This translates to efficiency in species origin identification, or rapid identification of species origin(s). If there is only one species origin, e.g. cattle, of the sample, then positive hybridization results will be detected from the hybridization(s) in which the DNA probe(s) is(are) designed from the DNA sequence of that particular species, i.e. cattle. On the other hand, if there are two species origins, e.g. cattle and pig, then positive hybridization results will be detected from the hybridizations in which the DNA probes are designed from the DNA sequences of both the species of cattle and pig. This provides a further technical advantage of allowing rapid identification of multiple species origins in one step. In other words, the present invention allows identification of mammalian species in a mixed population of samples within a single experiment.

As explained above, the usefulness of DNA probes will largely depend on the particular sequence thereof. The embodiment of the present invention provides a method engineering DNA probes for use in identification, but in the context of rapid identification of a mammalian species origin or mammalian species origins as explained above. The more detailed procedures for designing these DNA probes will then follow.

The DNA sequences used to define those probes are required to possess sufficient differences to distinguish between different species. In a further embodiment, the COI gene of a number of mammalian species is utilized. Specifically, studies leading to the present invention have shown that the 48^{th} to 705^{th} bp of this gene can be used as the DNA barcode for species identification in animals. The region can be amplified by PCR with the use of universal primers cocktail, making it available to generate a large amount of amplicons for hybridization for identification. This embodiment of the present invention will thus make use of this region.

Apart from the region of DNA sequence, to design species-specific DNA probes which are able to be used in a single experiment for identifying multiple species, those probes should have highly similar physical and chemical properties for hybridizing at the same environmental conditions. Moreover, other criteria are required to maximize the specificity of those probes. Therefore, in the present invention, criteria concerned for defining those probes include the length, guanine-cytosine (GC) content, delta G, melting temperature and hybridization temperature, and presence of secondary structure.

There is however priority in these criteria when defining those probes. Length generally is the first concerned criterion. In theory, and typically, longer probe will give higher specificity and more efficient during hybridization although longer and yet reliable probe would be more difficult to generate. For example, synthesizing longer oligonucleotides would require enzymatic ligation approach which is more complicated and costly, rendering synthesis of longer probes commercially not possible in companies with less financial resources. Setting aside cost, cross hybridization is also related to the length of the oligonucleotide and is not desirable. Shorter probe will be easier to generate although their specificity will be weaker.

Of course the length of DNA probes depend on the particular type of application, in the context of this invention, studies leading to the present invention in the present context have identified that a length from 50-80 bp will be able to achieve the identification satisfactorily. Studies have shown that DNA probes with this length provide the ability to resolve species satisfactorily. In an alternative embodiment, the length of the DNA probes is from 60-80 bp. Based on the criteria discussed above, studies have shown that the present invention have defined probes within conventional DNA barcode region with length at 60, 70 and 80 bp for the mammalian species in Table 1.

The next criterion is GC content, i.e. the percentage of nitrogenous bases, guanine and cytosine, in the target sequence. The requirement on GC content differs, depending on the particular application. There are four different nitrogenous bases in DNA, including guanine (G), cytosine (C), adenine (A) and thymine (T). This might be due to the fact that G and C bind each other by forming 3 hydrogen bonds while A and T form 2 hydrogen bonds, making a DNA sequence with higher GC content more stable, or there could be other reasons. As a result, a higher GC content requires higher melting temperature (Tm) to provide enough energy for denaturing the DNA sequence. Moreover, higher hybridization temperature (T_{hyb}) for the DNA sequences is also resulted. While it has been suggested that the higher GC content increases the specificity in hybridization between oligonucleotide probe and complementary DNA, it does not mean the highest GC content of DNA probes will be suitable in the context of the present invention. If the GC content is too high, the DNA sequence could become undesirably coiled. Instead, according to the present invention, studies have shown that probes with GC content between 50% and 52% is appropriate for accuracy for the above listed mammalian species.

The third criterion is delta G which is the change in Gibbs Free Energy (in units of kcal/mole) between a system and the environment. In DNA or oligonucleotide sequence, when there are two complementary strands presence, they have tendency to form a duplex structure. A positive delta G at a given temperature indicates that the two strands tend to keep in single-stranded status while negative delta G indicates that the two stands tend to bind together to form a double-stranded structure. In the present invention, the DNA probes defined prefer to have positive delta G. The delta G used to define the probes at the hybridization temperature need larger than 1. Therefore, formation of hairpin structures due to self-binding of short complementary sequences within the probe sequence could be minimized.

Melting temperature (Tm) and hybridization temperature (T_{hyb}) are the followed criteria. These two criteria are inter-related. It has been shown that at Tm, 50% of DNA exists in single-stranded status, and there is mathematical equation or simulation for Tm. Generally, the equation derived by thermodynamic basis sets for nearest neighbor interactions is commonly used. On the other hand, hybridization conditions are selected to favor the formation of DNA-probe duplex. Usually, or at least in this embodiment, the hybridization temperature (T_{hyb}) is as much as 25° C. below the Tm, hence allowing the defined probes to bind to their complementary region on the amplicons. Moreover, the Tm is greatly affected by the GC content, length of the sequence and chemistry conditions, such as salt concentration and presence of organic solvents. Accordingly, in this embodiment the hybridization temperature (Thyb) substantially 25° C. below the melting temperature (Tm) is appropriate in allowing and enhancing the hybridization, and thus identification. (However, studies have shown that the hybridization temperature (T_{hyb}) may be from 15-25° C. below the Tm.) The melting temperature for the probes with GC content at 50% to 52% is between 79° C. to 83° C. Together with the criterion of delta G, the calculated T_{hyb} of the defined probes require above 60° C. with 50 mmole of sodium chloride and without any organic solvent.

Another criterion concerned is the presence of secondary structure (SS). In single-stranded DNA, SS referred to the structures, hairpins as mentioned in the previous section, formed by self-binding of short complementary sequences within the probe. Secondary structures occurred in oligonucleotide probes will impair the performance of the probes by reducing hybridization efficiency and increasing probe to probe variability. SS of a given single stranded DNA can be calculated and represented in SS value and number of SS. Probe with a SS value close to the number of SS indicates that there is a higher propensity for a nitrogenous base in the probe to be single stranded in all predicted SS. In the present invention, studies have shown that the difference between the number of secondary structures (SS) and the value of secondary structure (SS) should be between 0 to 4. Preferably, the probes with smallest number of SS and SS value closest to the number of SS are taken into account.

TABLE 1
List of targeted species for defining species
specific probes in the present invention

| Common Name | Scientific Name |
|---|---|
| Cattle | Bos taurus |
| Pig | Sus scrofa |
| Sheep | Ovis aries |
| Horse | Equus caballus |
| Dog | Canis lupus |
| Mouse | Mus musculus |

COI gene sequences data, including all full, partial and fragment sequences, of the targeted species can be obtained from GenBank and BOLD databases. When designing the DNA probes, duplicated sequence records between these two databases were removed. After alignment, the 48^{th} to 705^{th} bp fragment from 5′ end of each record was extracted. For those species with reference sequence in GenBank database, that particular sequence was used as the template to define the species-specific probes. For those species without reference sequences in GenBank database, the most populated sequence in that species was used as the template to define the probes.

The GC content and Tm of all possible sequences with length of 60, 70 and 80 bp from the obtained template sequences were calculated. Those sequences with GC content between 50% and 52% were picked for next step. All delta Gs at 25° C. and 65° C. of these picked sequences were calculated with the use of online program, ZIPFOLD, DINAMELT server. Delta G value at 25° C. and 65° C. larger than −4 kcal/mol and 1 kcal/mol respectively were selected for further step. At the 3′ end, if sequences contained 3 or more contiguous G or C nucleotide within the last 5 nucleotides, those sequences were discarded. Selected sequences were then passed to another online program, DNA-folding form, The Mfold web server for calculating the SS value and predicting the secondary structures at 25° C. and 65° C. Those sequences with standard deviation smaller than 1 in the SS value and the number of secondary structure smaller than 3 were selected. If no sequence fulfilled these criteria, those DNA sequences with values closest to these two criteria could be accepted.

After passing the selection with those physical criteria, the specificity of these selected sequences was verified within species and with other species. For intraspecific comparison, the selected sequences were compared with all COI genes of the same species obtained. Any different sequences found were also picked to undergo interspecific comparison. In such situation, a group of probes was defined for identifying that target species. If no difference was found, the sequences can be passed directly to interspecific comparison which was carried out by the BLAST system with GenBank database. In interspecific comparison, results are sorted according to the identity. Sequences that showed any result with identity higher than 97% to other species would not be used as species-specific probes.

Similar procedures described above can be applied for defining species specific probes for other organisms.

In order to appreciate the above novel method for rapid identification of a mammalian species origin or mammalian species origins according to the present invention is to be contrasted with two main conventional approaches, as follows.

### (a) DNA Fingerprinting by Restriction Fragment Length Polymorphism (RFLP)

This method was first introduced in 1985 for human identification and was subsequently applied to identification of other organisms. PCR amplification of short tandem repeat (STR) and variable number of tandem repeats (VNTR) had developed to improve the method. Endonucleases are used to cut the specific restriction sites on the PCR amplified amplicons, generating a number of small fragments at different sizes. The species specific pattern of different fragments can be observed on agarose gel after electrophoresis or DNA chips. These two detection methods had already applied to identify fish species. The problem is that with this approach, sophisticated procedure or expensive equipment is required, such as southern blotting or DNA chip analyzing machine. Moreover, incomplete digestion may occur and intraspecific variations could alter the number of restriction sites on the sequence. Besides, if the sample contained different species, RFLP cannot distinguish all species within one experiment since the fragment pattern generated is combined by several species specific patterns. Multiple experiment or further analysis to resolve the merged patterns is required.

### (b) DNA Sequencing

Due to the accumulation of mutation in the genome, particular genes or DNA regions have enough differences to serve as a marker for species identification. The differences can be notified by sequencing and comparing the target DNA region in different species. With PCR amplification technique, the PCR-sequencing method, which now is recognized as DNA-barcoding, has been applied in species identification for several years. A 658 base pairs (bp) sequence in mitochondrial gene, cytochrome c oxidase I (COI), have examined extensively to identify wild range of animal taxa, including insects, aquatic animals, and birds. U.S. Food and Drug Administration accepted COI-based barcoding as one of the species identification methods for fish. A standard operating procedure (SOP) had been published by FDA after a formal single laboratory validation at Center for Food Safety and Applied Nutrition (CFSAN), FDA). This SOP is intended to replace the LIB No. 4420 now. This conventional DNA barcoding method requires analyzing the sequence of whole barcode. Its application is limited by high operational cost due to the requirement of equipment and time for data analysis. Furthermore, additional costs and steps are required after PCR amplification if samples containing multiple species are examining. PCR amplified amplicons with different species cannot be sequenced in a single experiment. Amplicon separation of different species by bacterial cloning is required. Processes involve ligation, transformation, culture of bacterial colonies on agar plate. Extra step, including picking bacterial colonies, culture of bacterial in broth or even prepping the amplicon ligated plasmids is required if the biotechnology company have not provide those services. It will further increase 2 more days in overall process.

In view of the conventional approaches, and to decrease the cost and shorten the time for species identification, and to further allow the identification of multiple species within one experiment, new identification approach is required. The present invention, as illustrated above, defines species specific DNA sequences served as DNA probes for identifying multiple species in the same experiment through DNA hybridization. The target species involved in the present invention are mammalian species, including Bos taurus, Sus scrofa, Ovis aries, Equus caballus, Canis lupus and Mus musculus.

It is to be noted that more than one probe may be needed to ascertain whether a sample originates from a particular inter-species or not. This is because in certain region of the COI gene of a particular inter-species, the sequence thereof may differ among intra-species of that particular inter-species. To assess whether the sample originates from the inter-species or not, multiple probes derived from different intra-sequences of all of the known intra-species would be needed. For example, probes with SEQ ID NOs. 7-11 shown in Appendix are all derived from different intra-species of the same inter-species. Accordingly, it is envisaged that the present invention would be capable of detecting not only the inter-species origin(s) of a sample, but also the intra-species origin(s) of a sample.

In order to more clearly illustrate the present invention, the following, with reference to the figures, will demonstrate the elements of the present invention by way of example.

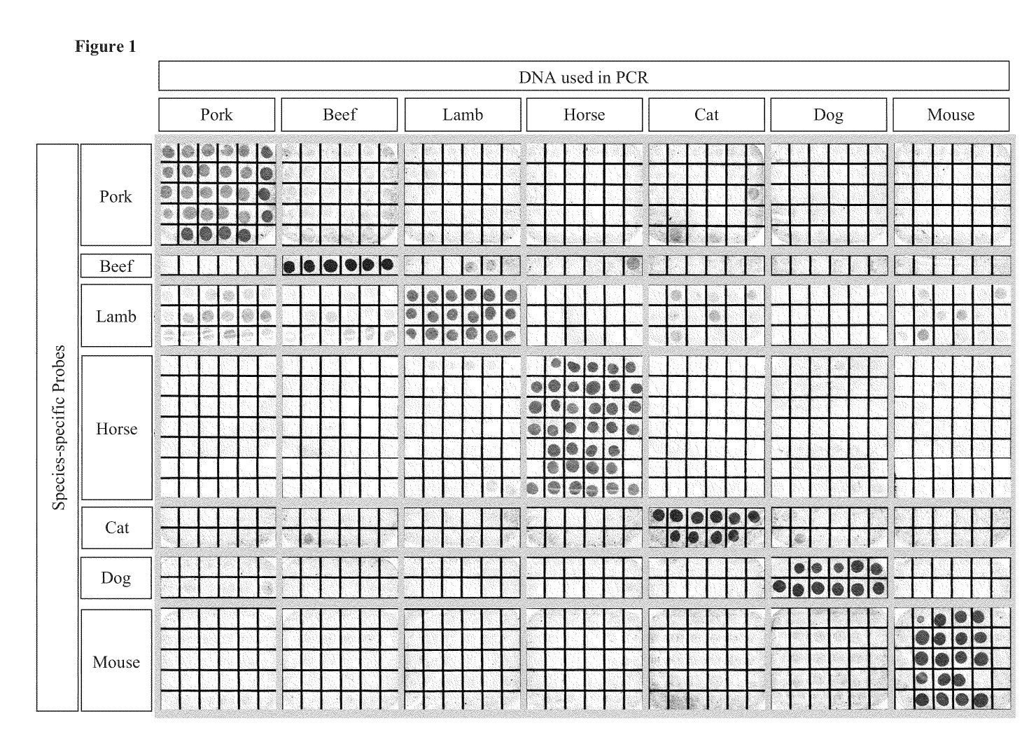

A first experiment was conduct to test whether the engineered DNA probes were able to identify mammalian species samples with sufficient species specificity. In this experiment, the DNA used in each PCR is isolated or originates from a single species. PCR amplicons of each species were hybridized to all probes as indicated in FIG. 1. Hybridization temperature was 42° C. 10 min color development for signal detection was applied. Signal was detected after hybridization between species-specific probes.

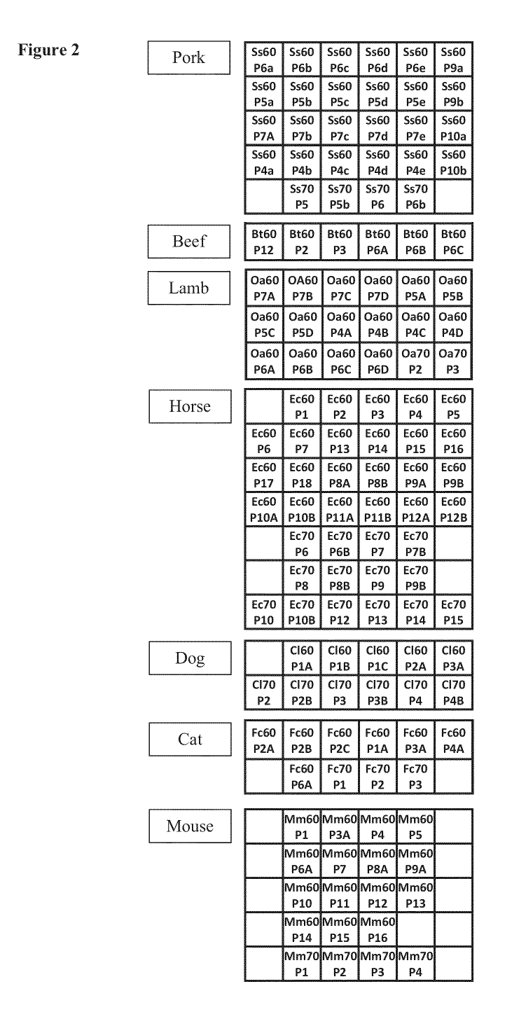

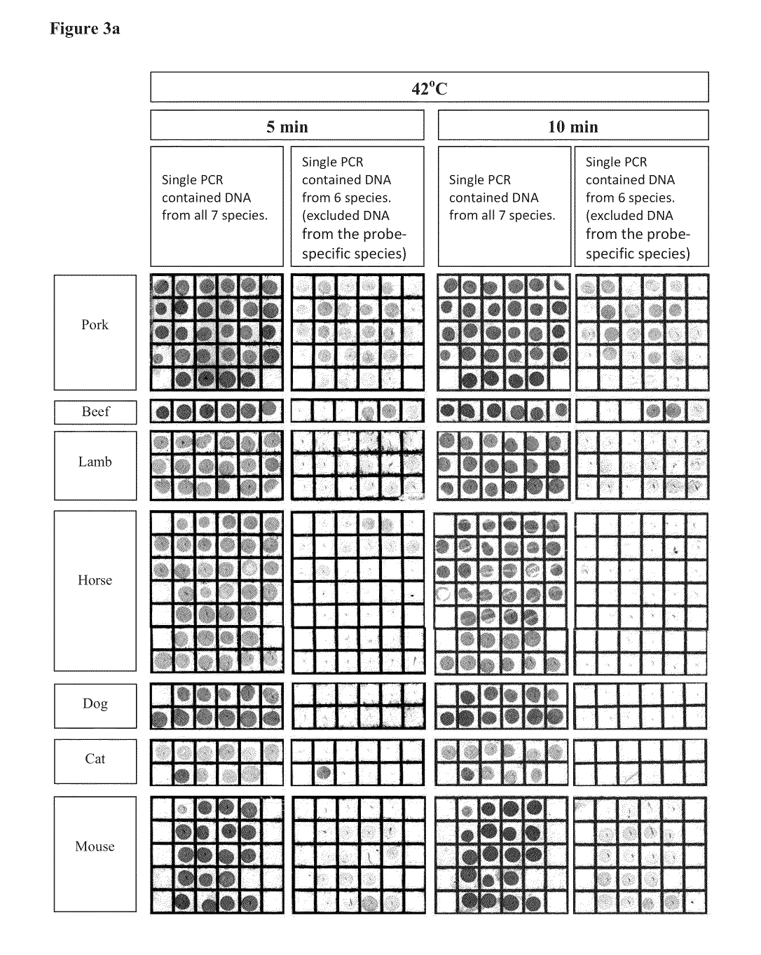

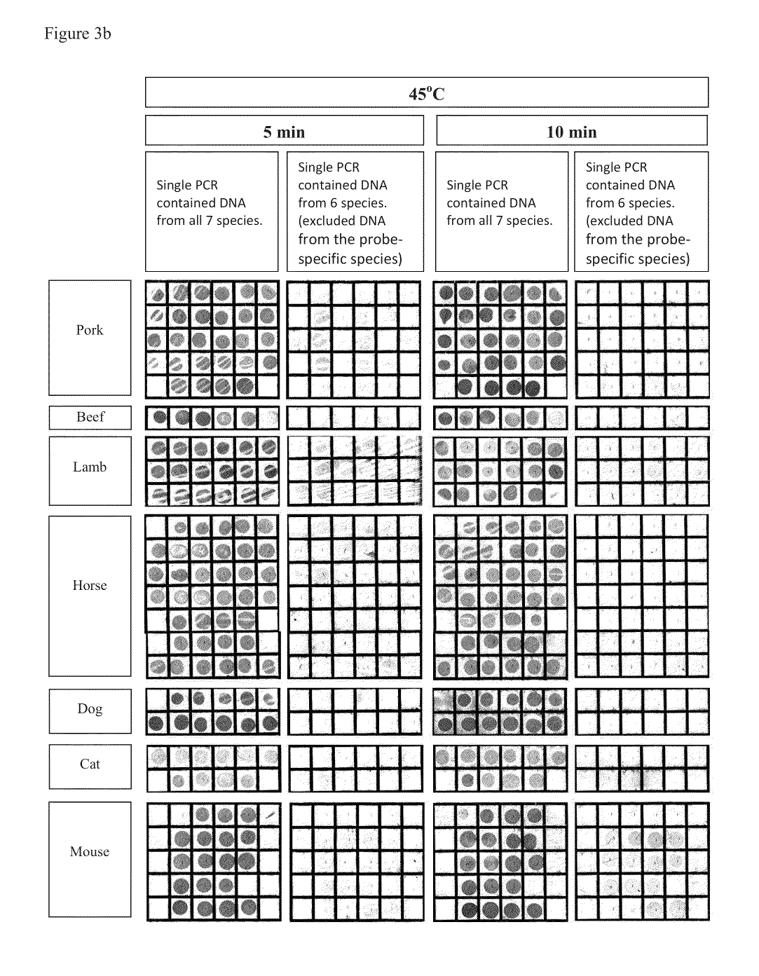

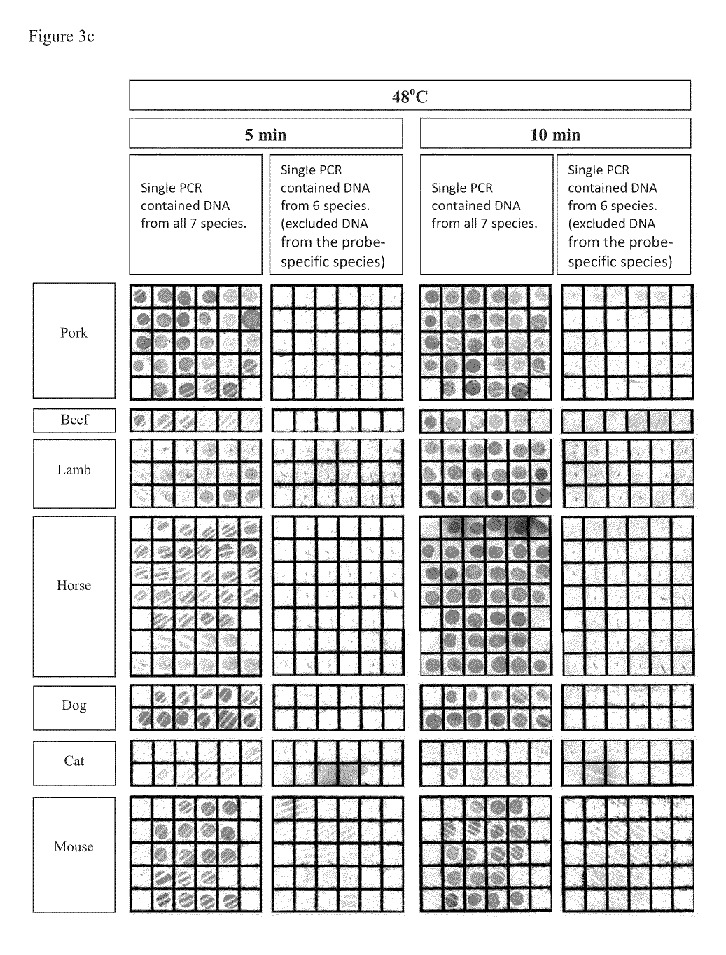

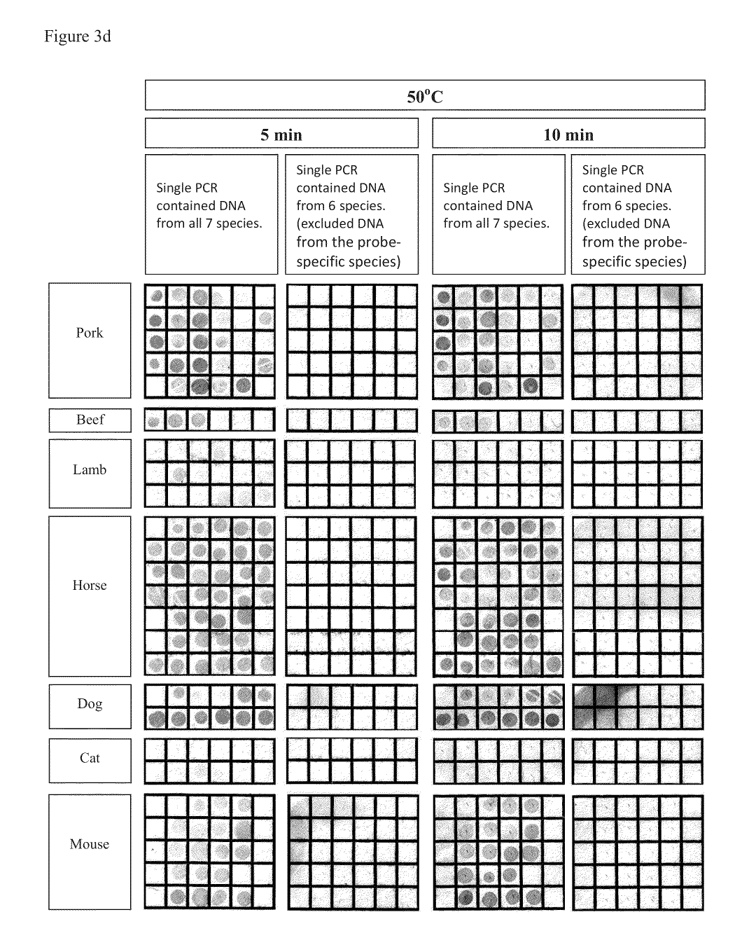

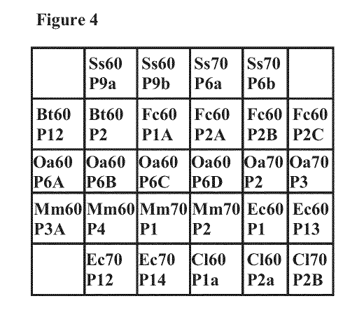

A number of observations can be seen from FIG. 1. Please however also refer to FIG. 2, FIG. 6 and the Sequence Listing. Specifically, FIG. 2 is a key showing which probes are located in which wells on the templates. The probes were immobilized on the membranes used in the experiments, results of which are shown in Figure, and FIGS. 3a to 3d. The following is also to be noted.
Ss: Sus scrofa (Pork)
Bt: Bos taurus (Beef)
Oa: Ovis aries (Lamb)
Ec: Equus caballus (Horse)
Cl: Canis lupus (Dog)
Fc: Felis catus (Cat)
Mm: Mus musculus (Mouse)

For beef, probe P6 showed weak non-specific binding with Ovis aries (lamb) and Equus caballus (horse).

For pork, weak non-specific binding signal to Bos taurus (beef) was found in some probes. Non-specific binding signal to Felis catus (cat) were also found in probe P10a (60 bp), and probe P5a and P6a (70 bp).

For horse, Probes P1 to P4 showed weak non-specific signal against Ovis aries (lamb) and Canis lupus (dog).

70 bp Weak non-specific binding signal were observed in P14 and P15 against Canis lupus.

For cat, only probe P6 with 60 bp in length showed non-specific binding with Bos taurus.

For dog, All probes are specific to Canis lupus.

For mouse, All probes were specific to Mus musculus (mouse). Weak non-specific binding signal were found in probes with 70 bp length, P3 and P4, against Canis lupus.

For lamb, Probes P4a-4d, P5d, P6a-d and P7d in 60 bp length showed non-specific binding signal to Sus scrofa (pork). Probes P4b, P5b, P6b and P7b also showed non-specific binding signal to Felis catis and Mus musculus.

In another series of experiments, temperature for hybridization and time for hybridization for the identification of the species using the probes were studied.

In FIG. 3a, it is shown that the temperature for hybridization was at 42° C. Some non-specific signal was found in pork, beef, horse, cat and mouse specific probes. However, at least 2 to 3 probes in each species were specific enough at these hybridization conditions. Stronger signal was found when color development time was increased from 5 min to 10 min.

In FIG. 3b, the temperature for hybridization was increased from 42° C. to 45° C. Signal had become more specific. Nearly all probes were specific at 5 min color development time. Stronger signal were found when color development (hybridization) time was increased from 5 min to 10 min, but some non-specific signals were found in several mouse specific probes.

In FIG. 3c, signal became weaker when temperature was increased to 48° C. The cat specific probes nearly gave no signal. Apart from the cat specific probes, strong and specific signals were found in other species when color development time was increased to 10 min.

In FIG. 3d, signal became weak at 50° C. Nearly no signal could be detected for lamb and cat specific probes. However, most probes for pork, beef, horse, dog and mouse still gave specific signal.

In the experiments as shown in FIGS. 3a to 3d, it can be seen that the specific engineered probes are specific enough, although the working temperature for each species for optimal identification may vary. From all hybridization conditions, the hybridization temperature of about 45° C. yields better specific signal for all species. Several probes can be selected at this condition and place in the same membrane for a multi-species detection. The duration for color development is between about 5 to 10 min.

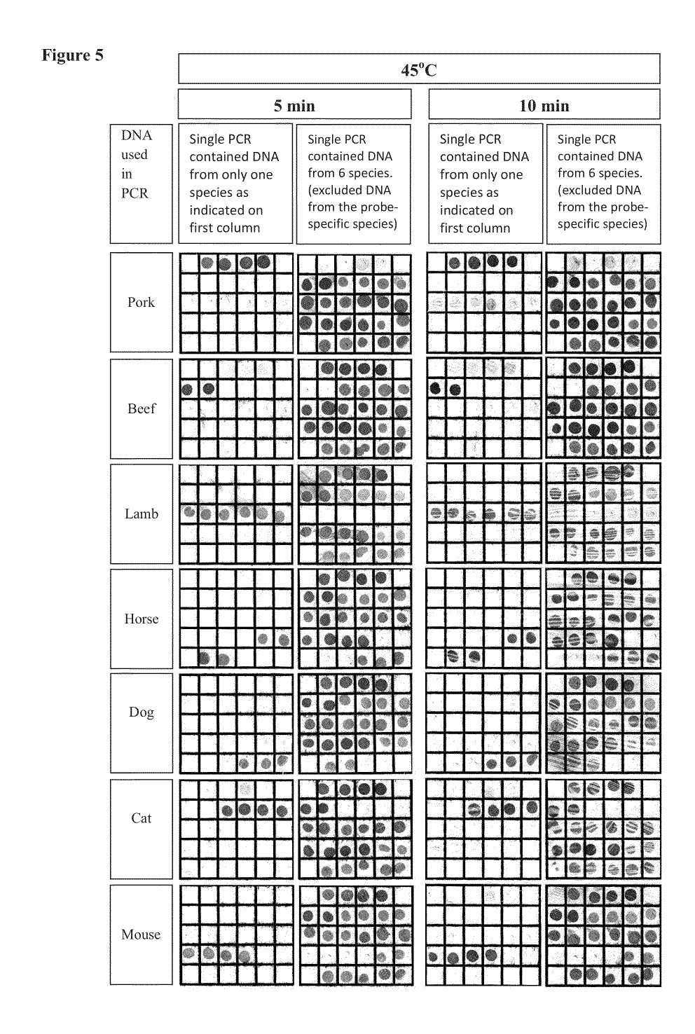

In another experiment, it is shown that the present invention can be applied for multiple species detection simultaneously. Referring to FIG. 5, there is shown a panel of species specific probes selected from each mammal species that are immobilized on the same membrane for multiple species detection. Please refer to FIG. 2 for the locations of the different probes in the panel.

Specific probes from each species were selected and immobilized on same membrane. The specificity of these probes in mixed samples was examined. For 5 min color development (hybridization time), it is shown that the engineered probes are specific to their own species. No non-specific signals were found when hybridized with PCR product amplified from DNA of other species. For 10 min of color development, signal was stronger but some non-specific signals were found although the identification was still satisfactory.

It is to be noted that the following a skilled person in the art possess the skills disclosed in the following reference, which are incorporated in this description in their entirety.
Gill, P., Jeffreys, A. J. and Werrett, D. J., (1985), Forensic application of DNA ‘fingerprints’. Nature, 12-18; 318 (6046): p 577-579.
Lockley, A. K. and Bardsley, R. G., (2000), DNA-based methods for food authentication. Trends in Food Science and Technology, 11, p 67-77.
Handy, S. M., Deeds, J. R., Ivanova, N. V., Hebert, P. D. N., Hanner, R., Ormos, A., Weigt, L. A., Moore, M. M. and Yancy, H. F. (2011). A single laboratory validated method for the generation of DNA barcodes for the identification of fish for regulatory compliance. Journal of AOAC International. 94 (1), 201-210.
Meusnier, I., Singer, G. A., Landry, J. F., Hickey, D. A., Hebert, P. D. and Hajibabaei, M., (2011), A universal DNA mini-barcode for biodiversity analysis, BMC Genomics, 9, p 214.
Shokralla, S., Zhou, X., Janzen, D. H., Hallwachs, W., Landry, J. F., Jacobus, L. M. and Hajibabaei, M., (2011), Pyrosequencing for mini-barcoding of fresh and old museum specimens, PLoS One, 6(7):e21252, Epub 2011 Jul. 27.
Chou, C. C., Chen, C. H., Lee, T. T. and Peck, K., (2004), Optimization of probe length and the number of probes per gene for optimal microarray analysis of gene expression, Nucleic Acids Research, 32(12), e99.
Aquino de Muro, M., (2008), Probe Design, Production, and Applications, Molecular

Biomethods Handbook, A, p 41-53.

It should be understood that the above only describes the preferred embodiments according to the present invention, and that modifications and alterations may be made thereto without departing from the spirit of the invention.

1. A method of rapid identification of a mammalian species origin or mammalian species origins of a sample, comprising the steps of:
a) gathering a sample to be tested;
b) processing the sample for use for identification purpose;
c) dividing the sample into a number of portions for situation in a multi-well container or containers;
d) providing a plurality of DNA probes, at least some of the DNA probes having DNA sequences with SEQ ID NOs. 1-241, wherein the number of the sample portions is greater than the number of mammalian species types from which the DNA probes derive;
e) selecting some or all of the DNA probes for hybridization with the sample portions;
f) subjecting the sample portions for intended hybridization with the selected DNA probes simultaneously;
g) detecting for positive hybridization results from the intended hybridization of the sample portions and the selected DNA probes; and
h) assessing the mammalian species origin or origins of the sample according to positive hybridization results.

2. A method as claimed in claim 1, wherein in step e) setting temperature for hybridization from 42-48° C.

3. A kit for use in rapid identification of mammalian species origin or mammalian species origins of a sample, comprising a plurality of DNA probes derived from a number of different mammalian species, at least some of the DNA probes having DNA sequences with SEQ ID NOs. 1-241.

4. A method of engineering DNA probes for use in rapid identification of mammalian species origin or mammalian species origins of a sample, comprising the steps of:
a) identifying regions of DNA sequence with a length from 60 to 80 bases from 48^{th} to 705^{th} bp of the COI gene of a first mammalian species, yielding a first group of DNA regions; and
b) from the first group of DNA regions, identifying regions of DNA sequence meeting the following criteria:
i) with GC content from substantially 50 to 52%;
ii) with a positive value of delta G, yielding a third group of DNA regions;
iii) in which the hybridization temperature (Thyb) is y from 15-25° C. below the melting temperature (Tm); and
iv) in which the difference between the number of secondary structures (SS) and the value of secondary structure (SS) is between 0 to 4; and
v) engineering DNA probes based on the identified DNA regions from said step b).

5. A method as claimed in claim 4, wherein the delta G value is larger than 1.

6. A method as claimed in claim 4, wherein the Tm is substantially 79-83° C.

7. A method as claimed in claim 4, wherein with 50 mmole of sodium chloride and without any organic solvent the Thyb is larger than 60° C.

8. A method as claimed in claim 4, wherein at least some of the DNA probes have DNA sequences with SEQ ID NOs. 1-241.

9. A method of engineering DNA probes for use in rapid identification of mammalian species origin or mammalian species origins of a sample, comprising the steps of:
a) identifying regions of DNA sequence with a length from 60 to 80 bases from 48^{th} to 705^{th} bp of the COI gene of a first mammalian species, thus yielding a first group of DNA regions;
b) from the first group of DNA regions, selecting regions of DNA sequence with GC content from substantially 50 to 52%, yielding a second group of DNA regions;
c) from the second group of DNA regions, selecting regions of DNA sequence with a positive value of delta G, yielding a third group of DNA regions;
d) from the third group of DNA regions, selecting regions of DNA sequence in which the hybridization temperature (Thyb) is 15-25° C. below the melting temperature (Tm), yielding a fourth group of DNA regions;
e) from the fourth group of DNA regions, selecting regions of DNA sequence in which the difference between the number of secondary structures (SS) and the value of secondary structure (SS) is between 0 to 4; and
f) engineering DNA probes based on the identified DNA regions from said step f).

10. A method as claimed in claim 9, wherein the delta G value is larger than 1.

11. A method as claimed in claim 9, wherein the Tm is substantially 79-83° C.

12. A method as claimed in claim 9, wherein with 50 mmole of sodium chloride and without any organic solvent the Thyb is larger than 60° C., NaCl w/o any organic solvent.

13. A method as claimed in claim 9, wherein at least some of the DNA probes have DNA sequences with SEQ ID NOs. 1-241.


SEQUENCE LISTING (241 sequences)

SEQ ID NO: 1 - DNA, 60 bp, Artificial Sequence
  Description of Artificial Sequence Synthetic probe
tccgtaataattaccgccgtactactactactctcgctccctgtattagcagccggcatc

SEQ ID NO: 2 - DNA, 60 bp, Artificial Sequence
  Description of Artificial Sequence Synthetic probe
ccgtaataattaccgccgtactactactactctcgctccctgtattagcagccggcatca

SEQ ID NO: 3 - DNA, 60 bp, Artificial Sequence
  Description of Artificial Sequence Synthetic probe
cgtaataattaccgccgtactactactactctcgctccctgtattagcagccggcatcac

SEQ ID NO: 4 - DNA, 60 bp, Artificial Sequence
  Description of Artificial Sequence Synthetic probe
ccggaacctaaatacaaccttcttcgacccggcaggaggaggagaccctattctatatca

SEQ ID NO: 5 - DNA, 60 bp, Artificial Sequence
  Description of Artificial Sequence Synthetic probe
ccgaaacctaaatacaaccttcttcgacccggcaggaggaggagaccctattctatatca

SEQ ID NO: 6 - DNA, 60 bp, Artificial Sequence
  Description of Artificial Sequence Synthetic probe
ccggaacctaaatacaactttcttcgacccggcaggaggaggagatcctattctatacca

SEQ ID NO: 7 - DNA, 60 bp, Artificial Sequence
  Description of Artificial Sequence Synthetic probe
agcctactaattcgcgctgaactaggtcagcccggaaccctacttggcgatgatcaaatc

SEQ ID NO: 8 - DNA, 60 bp, Artificial Sequence
  Description of Artificial Sequence Synthetic probe
agcctactaattcgcgctgaactaggtcagcccggaactctacttggcgatgatcaaatc

SEQ ID NO: 9 - DNA, 60 bp, Artificial Sequence
  Description of Artificial Sequence Synthetic probe
agcctgctaattcgcgctgaactaggtcagcccggaaccctacttggcgatgatcaaatc

SEQ ID NO: 10 - DNA, 60 bp, Artificial Sequence
  Description of Artificial Sequence Synthetic probe
agcctactaattcgcgctgaactaggtcagcccggaaccctacttccccatgatcaaatc

SEQ ID NO: 11 - DNA, 60 bp, Artificial Sequence
  Description of Artificial Sequence Synthetic probe
agcctactaattcgcgctgaactaggtcagcccggaactttacttggcgatgaccaaatc

SEQ ID NO: 12 - DNA, 60 bp, Artificial Sequence
  Description of Artificial Sequence Synthetic probe
ttgagcctactaattcgcgctgaactaggtcagcccggaaccctacttggcgatgatcaa

SEQ ID NO: 13 - DNA, 60 bp, Artificial Sequence
  Description of Artificial Sequence Synthetic probe
ttgagcctactaattcgcgctgaactaggtcagcccggaactctacttggcgatgatcaa

SEQ ID NO: 14 - DNA, 60 bp, Artificial Sequence
  Description of Artificial Sequence Synthetic probe
ttgagcctgctaattcgcgctgaactaggtcagcccggaaccctacttggcgatgatcaa

SEQ ID NO: 15 - DNA, 60 bp, Artificial Sequence
  Description of Artificial Sequence Synthetic probe
ttgagcctactaattcgcgctgaactaggtcagcccggaaccctacttccccatgatcaa

SEQ ID NO: 16 - DNA, 60 bp, Artificial Sequence
  Description of Artificial Sequence Synthetic probe
ttaagcctactaattcgcgctgaactaggtcagcccggaactttacttggcgatgaccaa

SEQ ID NO: 17 - DNA, 60 bp, Artificial Sequence
  Description of Artificial Sequence Synthetic probe
tgagcctactaattcgcgctgaactaggtcagcccggaaccctacttggcgatgatcaaa

SEQ ID NO: 18 - DNA, 60 bp, Artificial Sequence
  Description of Artificial Sequence Synthetic probe
tgagcctactaattcgcgctgaactaggtcagcccggaactctacttggcgatgatcaaa

SEQ ID NO: 19 - DNA, 60 bp, Artificial Sequence
  Description of Artificial Sequence Synthetic probe
tgagcctgctaattcgcgctgaactaggtcagcccggaaccctacttggcgatgatcaaa

SEQ ID NO: 20 - DNA, 60 bp, Artificial Sequence
  Description of Artificial Sequence Synthetic probe
tgagcctactaattcgcgctgaactaggtcagcccggaaccctacttccccatgatcaaa

SEQ ID NO: 21 - DNA, 60 bp, Artificial Sequence
  Description of Artificial Sequence Synthetic probe
taagcctactaattcgcgctgaactaggtcagcccggaactttacttggcgatgaccaaa

SEQ ID NO: 22 - DNA, 60 bp, Artificial Sequence
  Description of Artificial Sequence Synthetic probe
gagcctactaattcgcgctgaactaggtcagcccggaaccctacttggcgatgatcaaat

SEQ ID NO: 23 - DNA, 60 bp, Artificial Sequence
  Description of Artificial Sequence Synthetic probe
gagcctactaattcgcgctgaactaggtcagcccggaactctacttggcgatgatcaaat

SEQ ID NO: 24 - DNA, 60 bp, Artificial Sequence
  Description of Artificial Sequence Synthetic probe
gagcctgctaattcgcgctgaactaggtcagcccggaaccctacttggcgatgatcaaat

SEQ ID NO: 25 - DNA, 60 bp, Artificial Sequence
  Description of Artificial Sequence Synthetic probe
gagcctactaattcgcgctgaactaggtcagcccggaaccctacttccccatgatcaaat

SEQ ID NO: 26 - DNA, 60 bp, Artificial Sequence
  Description of Artificial Sequence Synthetic probe
aagcctactaattcgcgctgaactaggtcagcccggaactttacttggcgatgaccaaat

SEQ ID NO: 27 - DNA, 60 bp, Artificial Sequence
  Description of Artificial Sequence Synthetic probe
aagccggagcgggtactggatgaactgtatacccacctttagctggaaacttagcccatg

SEQ ID NO: 28 - DNA, 60 bp, Artificial Sequence
  Description of Artificial Sequence Synthetic probe
aagccggggcgggtactggatgaaccgtatacccacctttagctggaaacttagcccatg

SEQ ID NO: 29 - DNA, 60 bp, Artificial Sequence
  Description of Artificial Sequence Synthetic probe
aagccggggcgggtactggatgaaccgtatacccacctttagccggaaacttagcccatg

SEQ ID NO: 30 - DNA, 60 bp, Artificial Sequence
  Description of Artificial Sequence Synthetic probe
aggccggagcgggtactggatgaactgtatacccgcctttagctggaaacttagcccacg

SEQ ID NO: 31 - DNA, 60 bp, Artificial Sequence
  Description of Artificial Sequence Synthetic probe
aggccggagcgggtactggatgaactgtatacccacctttagctggaaacttagcccacg

SEQ ID NO: 32 - DNA, 60 bp, Artificial Sequence
  Description of Artificial Sequence Synthetic probe
aagccggagcgggtactggatgaaccgtatacccacctttagctggaaacttagcccatg

SEQ ID NO: 33 - DNA, 60 bp, Artificial Sequence
  Description of Artificial Sequence Synthetic probe
aagccggggcgggcactggatgaaccgtatacccacctttagctggaaacttagcccatg

SEQ ID NO: 34 - DNA, 60 bp, Artificial Sequence
  Description of Artificial Sequence Synthetic probe
aagccggggcgggtaccggatgaaccgtatacccacctttagctggaaacttagcccatg

SEQ ID NO: 35 - DNA, 60 bp, Artificial Sequence
  Description of Artificial Sequence Synthetic probe
aagccggggcgggtactggatgagccgtatacccacctttagctggaaacttagcccatg

SEQ ID NO: 36 - DNA, 60 bp, Artificial Sequence; identical to SEQ ID NO: 32
  Description of Artificial Sequence Synthetic probe

SEQ ID NO: 37 - DNA, 60 bp, Artificial Sequence
  Description of Artificial Sequence Synthetic probe
aagccggggcgggtactggatgaaccgtatacccacctttagctggaaacttagcccgtg

SEQ ID NO: 38 - DNA, 60 bp, Artificial Sequence
  Description of Artificial Sequence Synthetic probe
aagccggagcgggtactggatgaactgtatatccacctttagctggaaacttggcccatg

SEQ ID NO: 39 - DNA, 60 bp, Artificial Sequence
  Description of Artificial Sequence Synthetic probe
aagccggggcgggtactggatgaaccgaatacccacccttagctggaaacttagcccaag

SEQ ID NO: 40 - DNA, 60 bp, Artificial Sequence
  Description of Artificial Sequence Synthetic probe
aagccggggcgggtactggatgaaccgtatacccacctttagctggaaacttaccccatg

SEQ ID NO: 41 - DNA, 60 bp, Artificial Sequence
  Description of Artificial Sequence Synthetic probe
ggcatcctcaatagtagaagccggagcgggtactggatgaactgtatacccacctttagc

SEQ ID NO: 42 - DNA, 60 bp, Artificial Sequence
  Description of Artificial Sequence Synthetic probe
ggcatcctcaatagtagaagccggggcgggtactggatgaaccgtatacccacctttagc

SEQ ID NO: 43 - DNA, 60 bp, Artificial Sequence
  Description of Artificial Sequence Synthetic probe
ggcatcctcaatagtagaagccggggcgggcactggatgaaccgtatacccacctttagc

SEQ ID NO: 44 - DNA, 60 bp, Artificial Sequence
  Description of Artificial Sequence Synthetic probe
ggcatcctcaatagtagaagccggggcgggtactggatgaaccgaatacccacccttagc

SEQ ID NO: 45 - DNA, 60 bp, Artificial Sequence
  Description of Artificial Sequence Synthetic probe
ggcatcctcaatagtaaaagccggggcgggtactggatgaaccgtatacccacctttagc

SEQ ID NO: 46 - DNA, 60 bp, Artificial Sequence
  Description of Artificial Sequence Synthetic probe
ggcatcctcaatagtagaagccggggcgggtaccggatgaaccgtatacccacctttagc

SEQ ID NO: 47 - DNA, 60 bp, Artificial Sequence
  Description of Artificial Sequence Synthetic probe
ggcatcctcaatagtagaagccggagcgggtactggatgaaccgtatacccacctttagc

SEQ ID NO: 48 - DNA, 60 bp, Artificial Sequence
  Description of Artificial Sequence Synthetic probe
ggcatcctcaatagtagaagccggggcgggtactggatgagccgtatacccacctttagc

SEQ ID NO: 49 - DNA, 60 bp, Artificial Sequence
  Description of Artificial Sequence Synthetic probe
ggcatcctcaatagtagaagccggagcgggtactggatgaactgtatatccacctttagc

SEQ ID NO: 50 - DNA, 60 bp, Artificial Sequence
  Description of Artificial Sequence Synthetic probe
ggcatcctcaatagtagaggccggagcgggtactggatgaactgtatacccgcctttagc

SEQ ID NO: 51 - DNA, 60 bp, Artificial Sequence
  Description of Artificial Sequence Synthetic probe
ggcatcctcaatagtagaggccggagcgggtactggatgaactgtatacccacctttagc

SEQ ID NO: 52 - DNA, 60 bp, Artificial Sequence
  Description of Artificial Sequence Synthetic probe
ggcgtcctcaatagtagaagccggagcgggtactggatgaactgtatacccacctttagc

SEQ ID NO: 53 - DNA, 70 bp, Artificial Sequence
  Description of Artificial Sequence Synthetic probe
caatagtagaagccggagcgggtactggatgaactgtatacccacctttagctggaaacttagcccatgc

SEQ ID NO: 54 - DNA, 70 bp, Artificial Sequence
  Description of Artificial Sequence Synthetic probe
caatagtagaagccggggcgggtactggatgaaccgtatacccacctttagctggaaacttagcccatgc

SEQ ID NO: 55 - DNA, 70 bp, Artificial Sequence
  Description of Artificial Sequence Synthetic probe
caatagtagaagccggagcgggtactggatgaaccgtatacccacctttagctggaaacttagcccatgc

SEQ ID NO: 56 - DNA, 70 bp, Artificial Sequence
  Description of Artificial Sequence Synthetic probe
caatagtagaggccggagcgggtactggatgaactgtatacccacctttagctggaaacttagcccacgc

SEQ ID NO: 57 - DNA, 70 bp, Artificial Sequence
  Description of Artificial Sequence Synthetic probe
caatagtagaggccggagcgggtactggatgaactgtatacccgcctttagctggaaacttagcccacgc

SEQ ID NO: 58 - DNA, 70 bp, Artificial Sequence
  Description of Artificial Sequence Synthetic probe
caatagtagaagccggggcgggtactggatgagccgtatacccacctttagctggaaacttagcccatgc

SEQ ID NO: 59 - DNA, 70 bp, Artificial Sequence
  Description of Artificial Sequence Synthetic probe
caatagtagaagccggggcgggcactggatgaaccgtatacccacctttagctggaaacttagcccatgc

SEQ ID NO: 60 - DNA, 70 bp, Artificial Sequence
  Description of Artificial Sequence Synthetic probe
caatagtagaagccggggcgggtactggatgaaccgtatacccacctttagctggaaacttaccccatgc

SEQ ID NO: 61 - DNA, 70 bp, Artificial Sequence
  Description of Artificial Sequence Synthetic probe
caatagtagaagccggggcgggtactggatgaaccgaatacccacccttagctggaaacttagcccaagc

SEQ ID NO: 62 - DNA, 70 bp, Artificial Sequence
  Description of Artificial Sequence Synthetic probe
caatagtagaagccggggcgggtactggatgaaccgtatacccacctttagctggaaacttagcccgtgc

SEQ ID NO: 63 - DNA, 70 bp, Artificial Sequence
  Description of Artificial Sequence Synthetic probe
caatagtagaagccggggcgggtactggatgaaccgtatacccacctttagccggaaacttagcccatgc

SEQ ID NO: 64 - DNA, 70 bp, Artificial Sequence
  Description of Artificial Sequence Synthetic probe
caatagtagaagccggggcgggtaccggatgaaccgtatacccacctttagctggaaacttagcccatgc

SEQ ID NO: 65 - DNA, 70 bp, Artificial Sequence
  Description of Artificial Sequence Synthetic probe
caatagtaaaagccggggcgggtactggatgaaccgtatacccacctttagctggaaacttagcccatgc

SEQ ID NO: 66 - DNA, 70 bp, Artificial Sequence
  Description of Artificial Sequence Synthetic probe
caatagtagaagccggagcgggtactggatgaactgtatatccacctttagctggaaacttggcccatgc

SEQ ID NO: 67 - DNA, 70 bp, Artificial Sequence
  Description of Artificial Sequence Synthetic probe
atagtagaagccggagcgggtactggatgaactgtatacccacctttagctggaaacttagcccatgcag

SEQ ID NO: 68 - DNA, 70 bp, Artificial Sequence
  Description of Artificial Sequence Synthetic probe
atagtagaagccggggcgggtactggatgaaccgtatacccacctttagctggaaacttagcccatgcag

SEQ ID NO: 69 - DNA, 70 bp, Artificial Sequence
  Description of Artificial Sequence Synthetic probe
atagtagaggccggagcgggtactggatgaactgtatacccacctttagctggaaacttagcccacgcag

SEQ ID NO: 70 - DNA, 70 bp, Artificial Sequence
  Description of Artificial Sequence Synthetic probe
atagtagaggccggagcgggtactggatgaactgtatacccgcctttagctggaaacttagcccacgcag

SEQ ID NO: 71 - DNA, 70 bp, Artificial Sequence
  Description of Artificial Sequence Synthetic probe
atagtagaagccggggcgggtactggatgagccgtatacccacctttagctggaaacttagcccatgcag

SEQ ID NO: 72 - DNA, 70 bp, Artificial Sequence
  Description of Artificial Sequence Synthetic probe
atagtagaagccggggcgggcactggatgaaccgtatacccacctttagctggaaacttagcccatgcag

SEQ ID NO: 73 - DNA, 70 bp, Artificial Sequence
  Description of Artificial Sequence Synthetic probe
atagtagaagccggggcgggtactggatgaaccgtatacccacctttagctggaaacttaccccatgcag

SEQ ID NO: 74 - DNA, 70 bp, Artificial Sequence
  Description of Artificial Sequence Synthetic probe
atagtagaagccggggcgggtactggatgaaccgaatacccacccttagctggaaacttagcccaagcag

SEQ ID NO: 75 - DNA, 70 bp, Artificial Sequence
  Description of Artificial Sequence Synthetic probe
atagtagaagccggggcgggtactggatgaaccgtatacccacctttagctggaaacttagcccgtgcag

SEQ ID NO: 76 - DNA, 70 bp, Artificial Sequence
  Description of Artificial Sequence Synthetic probe
atagtagaagccggggcgggtactggatgaaccgtatacccacctttagccggaaacttagcccatgcag

SEQ ID NO: 77 - DNA, 70 bp, Artificial Sequence
  Description of Artificial Sequence Synthetic probe
atagtagaagccggggcgggtaccggatgaaccgtatacccacctttagctggaaacttagcccatgcag

SEQ ID NO: 78 - DNA, 70 bp, Artificial Sequence
  Description of Artificial Sequence Synthetic probe
atagtaaaagccggggcgggtactggatgaaccgtatacccacctttagctggaaacttagcccatgcag

SEQ ID NO: 79 - DNA, 70 bp, Artificial Sequence
  Description of Artificial Sequence Synthetic probe
atagtagaagccggagcgggtactggatgaactgtatatccacctttagctggaaacttggcccatgcag

SEQ ID NO: 80 - DNA, 60 bp, Artificial Sequence
  Description of Artificial Sequence Synthetic probe
gcccatgcaggagcctcagtagatctaactattttctccctacatctggcaggtgtctct

SEQ ID NO: 81 - DNA, 60 bp, Artificial Sequence
  Description of Artificial Sequence Synthetic probe
gcccatgcaggagcctcagtagacctaactattttctccctacacctggcaggtgtctct

SEQ ID NO: 82 - DNA, 60 bp, Artificial Sequence
  Description of Artificial Sequence Synthetic probe
gcccatgcaggagcctcagtagatctaattattttctccctacacctggcaggtgtctct

SEQ ID NO: 83 - DNA, 60 bp, Artificial Sequence
  Description of Artificial Sequence Synthetic probe
gcccatgcaggagcctcagtagatctaactattttctccctacacctggcaggtgtctct

SEQ ID NO: 84 - DNA, 60 bp, Artificial Sequence
  Description of Artificial Sequence Synthetic probe
agcccatgcaggagcctcagtagatctaactattttctccctacatctggcaggtgtctc

SEQ ID NO: 85 - DNA, 60 bp, Artificial Sequence
  Description of Artificial Sequence Synthetic probe
agcccatgcaggagcctcagtagacctaactattttctccctacacctggcaggtgtctc

SEQ ID NO: 86 - DNA, 60 bp, Artificial Sequence
  Description of Artificial Sequence Synthetic probe
agcccatgcaggagcctcagtagatctaattattttctccctacacctggcaggtgtctc

SEQ ID NO: 87 - DNA, 60 bp, Artificial Sequence
  Description of Artificial Sequence Synthetic probe
agcccatgcaggagcctcagtagatctaactattttctccctacacctggcaggtgtctc

SEQ ID NO: 88 - DNA, 60 bp, Artificial Sequence
  Description of Artificial Sequence Synthetic probe
ctagcccatgcaggagcctcagtagatctaactattttctccctacatctggcaggtgtc

SEQ ID NO: 89 - DNA, 60 bp, Artificial Sequence
  Description of Artificial Sequence Synthetic probe
ctagcccatgcaggagcctcagtagacctaactattttctccctacacctggcaggtgtc

SEQ ID NO: 90 - DNA, 60 bp, Artificial Sequence
  Description of Artificial Sequence Synthetic probe
ctagcccatgcaggagcctcagtagatctaattattttctccctacacctggcaggtgtc

SEQ ID NO: 91 - DNA, 60 bp, Artificial Sequence
  Description of Artificial Sequence Synthetic probe
ctagcccatgcaggagcctcagtagatctaactattttctccctacacctggcaggtgtc

SEQ ID NO: 92 - DNA, 60 bp, Artificial Sequence
  Description of Artificial Sequence Synthetic probe
cctagcccatgcaggagcctcagtagatctaactattttctccctacatctggcaggtgt

SEQ ID NO: 93 - DNA, 60 bp, Artificial Sequence
  Description of Artificial Sequence Synthetic probe
cctagcccatgcaggagcctcagtagacctaactattttctccctacacctggcaggtgt

SEQ ID NO: 94 - DNA, 60 bp, Artificial Sequence
  Description of Artificial Sequence Synthetic probe
cctagcccatgcaggagcctcagtagatctaattattttctccctacacctggcaggtgt

SEQ ID NO: 95 - DNA, 60 bp, Artificial Sequence
  Description of Artificial Sequence Synthetic probe
cctagcccatgcaggagcctcagtagatctaactattttctccctacacctggcaggtgt

SEQ ID NO: 96 - DNA, 70 bp, Artificial Sequence
  Description of Artificial Sequence Synthetic probe
gcaacctagcccatgcaggagcctcagtagatctaactattttctccctacatctggcaggtgtctcttc

SEQ ID NO: 97 - DNA, 70 bp, Artificial Sequence
  Description of Artificial Sequence Synthetic probe
gcaacctagcccatgcaggagcctcagtagatctaactattttctccctacacctggcaggtgtctcttc

SEQ ID NO: 98 - DNA, 70 bp, Artificial Sequence
  Description of Artificial Sequence Synthetic probe
gcaacctagcccatgcaggagcctcagtagatctaattattttctccctacacctggcaggtgtctcttc

SEQ ID NO: 99 - DNA, 70 bp, Artificial Sequence
  Description of Artificial Sequence Synthetic probe
gcaacctagcccatgcaggagcctcagtagacctaactattttctccctacacctggcaggtgtctcttc

SEQ ID NO: 100 - DNA, 70 bp, Artificial Sequence
  Description of Artificial Sequence Synthetic probe
ggcaacctagcccatgcaggagcctcagtagatctaactattttctccctacatctggcaggtgtctctt

SEQ ID NO: 101 - DNA, 70 bp, Artificial Sequence
  Description of Artificial Sequence Synthetic probe
ggcaacctagcccatgcaggagcctcagtagatctaactattttctccctacacctggcaggtgtctctt

SEQ ID NO: 102 - DNA, 70 bp, Artificial Sequence
  Description of Artificial Sequence Synthetic probe
ggcaacctagcccatgcaggagcctcagtagatctaattattttctccctacacctggcaggtgtctctt

SEQ ID NO: 103 - DNA, 70 bp, Artificial Sequence
  Description of Artificial Sequence Synthetic probe
ggcaacctagcccatgcaggagcctcagtagacctaactattttctccctacacctggcaggtgtctctt

SEQ ID NO: 104 - DNA, 60 bp, Artificial Sequence
  Description of Artificial Sequence Synthetic probe
tgaaccgtatatcctcctctagctggaaatctggcgcatgcaggagcctctgttgactta

SEQ ID NO: 105 - DNA, 60 bp, Artificial Sequence
  Description of Artificial Sequence Synthetic probe
gaaccgtatatcctcctctagctggaaatctggcgcatgcaggagcctctgttgacttaa

SEQ ID NO: 106 - DNA, 60 bp, Artificial Sequence
  Description of Artificial Sequence Synthetic probe
ctgaaccgtatatcctcctctagctggaaatctggcgcatgcaggagcctctgttgactt

SEQ ID NO: 107 - DNA, 60 bp, Artificial Sequence
  Description of Artificial Sequence Synthetic probe
aaccgtatatcctcctctagctggaaatctggcgcatgcaggagcctctgttgacttaac

SEQ ID NO: 108 - DNA, 60 bp, Artificial Sequence
  Description of Artificial Sequence Synthetic probe
cctctagctggaaatctggcgcatgcaggagcctctgttgacttaaccattttctctctc

SEQ ID NO: 109 - DNA, 60 bp, Artificial Sequence
  Description of Artificial Sequence Synthetic probe
ctctagctggaaatctggcgcatgcaggagcctctgttgacttaaccattttctctctcc

SEQ ID NO: 110 - DNA, 60 bp, Artificial Sequence
  Description of Artificial Sequence Synthetic probe
ctagctggaaatctggcgcatgcaggagcctctgttgacttaaccattttctctctccac

SEQ ID NO: 111 - DNA, 60 bp, Artificial Sequence
  Description of Artificial Sequence Synthetic probe
gctggaaatctggcgcatgcaggagcctctgttgacttaaccattttctctctccaccta

SEQ ID NO: 112 - DNA, 60 bp, Artificial Sequence
  Description of Artificial Sequence Synthetic probe
gctggaaatctggcgcatgcaggagcctctgttgacttaaccattttctctctccacctg

SEQ ID NO: 113 - DNA, 60 bp, Artificial Sequence
  Description of Artificial Sequence Synthetic probe
ctggaaatctggcgcatgcaggagcctctgttgacttaaccattttctctctccacctag

SEQ ID NO: 114 - DNA, 60 bp, Artificial Sequence
  Description of Artificial Sequence Synthetic probe
ctggaaatctggcgcatgcaggagcctctgttgacttaaccattttctctctccacctgg

SEQ ID NO: 115 - DNA, 60 bp, Artificial Sequence
  Description of Artificial Sequence Synthetic probe
tggaaatctggcgcatgcaggagcctctgttgacttaaccattttctctctccacctagc

SEQ ID NO: 116 - DNA, 60 bp, Artificial Sequence
  Description of Artificial Sequence Synthetic probe
tggaaatctggcgcatgcaggagcctctgttgacttaaccattttctctctccacctggc

SEQ ID NO: 117 - DNA, 60 bp, Artificial Sequence
  Description of Artificial Sequence Synthetic probe
ggaaatctggcgcatgcaggagcctctgttgacttaaccattttctctctccacctagct

SEQ ID NO: 118 - DNA, 60 bp, Artificial Sequence
  Description of Artificial Sequence Synthetic probe
ggaaatctggcgcatgcaggagcctctgttgacttaaccattttctctctccacctggct

SEQ ID NO: 119 - DNA, 60 bp, Artificial Sequence
  Description of Artificial Sequence Synthetic probe
gaaatctggcgcatgcaggagcctctgttgacttaaccattttctctctccacctagctg

SEQ ID NO: 120 - DNA, 60 bp, Artificial Sequence
  Description of Artificial Sequence Synthetic probe
gaaatctggcgcatgcaggagcctctgttgacttaaccattttctctctccacctggctg

SEQ ID NO: 121 - DNA, 60 bp, Artificial Sequence
  Description of Artificial Sequence Synthetic probe
tcctcctctagctggaaatctggcgcatgcaggagcctctgttgacttaaccattttctc

SEQ ID NO: 122 - DNA, 60 bp, Artificial Sequence
  Description of Artificial Sequence Synthetic probe
cctcctctagctggaaatctggcgcatgcaggagcctctgttgacttaaccattttctct

SEQ ID NO: 123 - DNA, 60 bp, Artificial Sequence
  Description of Artificial Sequence Synthetic probe
ctcctctagctggaaatctggcgcatgcaggagcctctgttgacttaaccattttctctc

SEQ ID NO: 124 - DNA, 60 bp, Artificial Sequence
  Description of Artificial Sequence Synthetic probe
cgtatatcctcctctagctggaaatctggcgcatgcaggagcctctgttgacttaaccat

SEQ ID NO: 125 - DNA, 60 bp, Artificial Sequence
  Description of Artificial Sequence Synthetic probe
accgtatatcctcctctagctggaaatctggcgcatgcaggagcctctgttgacttaacc

SEQ ID NO: 126 - DNA, 60 bp, Artificial Sequence
  Description of Artificial Sequence Synthetic probe
ccgtatatcctcctctagctggaaatctggcgcatgcaggagcctctgttgacttaacca

SEQ ID NO: 127 - DNA, 70 bp, Artificial Sequence
  Description of Artificial Sequence Synthetic probe
tatcctcctctagctggaaatctggcgcatgcaggagcctctgttgacttaaccattttctctctccacc

SEQ ID NO: 128 - DNA, 70 bp, Artificial Sequence
  Description of Artificial Sequence Synthetic probe
atcctcctctagctggaaatctggcgcatgcaggagcctctgttgacttaaccattttctctctccacct

SEQ ID NO: 129 - DNA, 70 bp, Artificial Sequence
  Description of Artificial Sequence Synthetic probe
ggctgaaccgtatatcctcctctagctggaaatctggcgcatgcaggagcctctgttgacttaaccattt

SEQ ID NO: 130 - DNA, 70 bp, Artificial Sequence
  Description of Artificial Sequence Synthetic probe
aggctgaaccgtatatcctcctctagctggaaatctggcgcatgcaggagcctctgttgacttaaccatt

SEQ ID NO: 131 - DNA, 70 bp, Artificial Sequence
  Description of Artificial Sequence Synthetic probe
cctctagctggaaatctggcgcatgcaggagcctctgttgacttaaccattttctctctccacctagctg

SEQ ID NO: 132 - DNA, 70 bp, Artificial Sequence
  Description of Artificial Sequence Synthetic probe
cctctagctggaaatctggcgcatgcaggagcctctgttgacttaaccattttctctctccacctggctg

SEQ ID NO: 133 - DNA, 70 bp, Artificial Sequence
  Description of Artificial Sequence Synthetic probe
tcctcctctagctggaaatctggcgcatgcaggagcctctgttgacttaaccattttctctctccaccta

SEQ ID NO: 134 - DNA, 70 bp, Artificial Sequence
  Description of Artificial Sequence Synthetic probe
tcctcctctagctggaaatctggcgcatgcaggagcctctgttgacttaaccattttctctctccacctg

SEQ ID NO: 135 - DNA, 70 bp, Artificial Sequence
  Description of Artificial Sequence Synthetic probe
tcctctagctggaaatctggcgcatgcaggagcctctgttgacttaaccattttctctctccacctagct

SEQ ID NO: 136 - DNA, 70 bp, Artificial Sequence
  Description of Artificial Sequence Synthetic probe
tcctctagctggaaatctggcgcatgcaggagcctctgttgacttaaccattttctctctccacctggct

SEQ ID NO: 137 - DNA, 70 bp, Artificial Sequence
  Description of Artificial Sequence Synthetic probe
cctcctctagctggaaatctggcgcatgcaggagcctctgttgacttaaccattttctctctccacctag

SEQ ID NO: 138 - DNA, 70 bp, Artificial Sequence
  Description of Artificial Sequence Synthetic probe
cctcctctagctggaaatctggcgcatgcaggagcctctgttgacttaaccattttctctctccacctgg

SEQ ID NO: 139 - DNA, 70 bp, Artificial Sequence
  Description of Artificial Sequence Synthetic probe
ctcctctagctggaaatctggcgcatgcaggagcctctgttgacttaaccattttctctctccacctagc

SEQ ID NO: 140 - DNA, 70 bp, Artificial Sequence
  Description of Artificial Sequence Synthetic probe
ctcctctagctggaaatctggcgcatgcaggagcctctgttgacttaaccattttctctctccacctggc

SEQ ID NO: 141 - DNA, 60 bp, Artificial Sequence
  Description of Artificial Sequence Synthetic probe
cctcctcatccgagccgaactaggtcagcccggtactttactaggtgacgatcaaattta

SEQ ID NO: 142 - DNA, 60 bp, Artificial Sequence
  Description of Artificial Sequence Synthetic probe
cctcctcatccgagccgaactaggtcagcccggtactttactaggtgatgatcaaattta

SEQ ID NO: 143 - DNA, 60 bp, Artificial Sequence
  Description of Artificial Sequence Synthetic probe
cctcctcatccgagccgaactaggtcagcccggtactttactaggcgacgatcaaattta

SEQ ID NO: 144 - DNA, 60 bp, Artificial Sequence
  Description of Artificial Sequence Synthetic probe
cctcctcatccgggccgaactaggtcagcccggtactttactaggtgacgatcaaattta

SEQ ID NO: 145 - DNA, 60 bp, Artificial Sequence
  Description of Artificial Sequence Synthetic probe
cctcctcatccgagccgaactaggtcagcccggtactttactaggagacgatcagattta

SEQ ID NO: 146 - DNA, 60 bp, Artificial Sequence
  Description of Artificial Sequence Synthetic probe
agcctcctcatccgagccgaactaggtcagcccggtactttactaggtgacgatcaaatt

SEQ ID NO: 147 - DNA, 60 bp, Artificial Sequence
  Description of Artificial Sequence Synthetic probe
agcctcctcatccgagccgaactaggtcagcccggtactttactaggtgatgatcaaatt

SEQ ID NO: 148 - DNA, 60 bp, Artificial Sequence
  Description of Artificial Sequence Synthetic probe
agcctcctcatccgagccgaactaggtcagcccggtactttactaggcgacgatcaaatt

SEQ ID NO: 149 - DNA, 60 bp, Artificial Sequence
  Description of Artificial Sequence Synthetic probe
agcctcctcatccgggccgaactaggtcagcccggtactttactaggtgacgatcaaatt

SEQ ID NO: 150 - DNA, 60 bp, Artificial Sequence
  Description of Artificial Sequence Synthetic probe
agcctcctcatccgagccgaactaggtcagcccggtactttactaggagacgatcagatt

SEQ ID NO: 151 - DNA, 60 bp, Artificial Sequence
  Description of Artificial Sequence Synthetic probe
gcctcctcatccgagccgaactaggtcagcccggtactttactaggtgacgatcaaattt

SEQ ID NO: 152 - DNA, 60 bp, Artificial Sequence
  Description of Artificial Sequence Synthetic probe
gcctcctcatccgagccgaactaggtcagcccggtactttactaggtgatgatcaaattt

SEQ ID NO: 153 - DNA, 60 bp, Artificial Sequence
  Description of Artificial Sequence Synthetic probe
gcctcctcatccgagccgaactaggtcagcccggtactttactaggcgacgatcaaattt

SEQ ID NO: 154 - DNA, 60 bp, Artificial Sequence
  Description of Artificial Sequence Synthetic probe
gcctcctcatccgggccgaactaggtcagcccggtactttactaggtgacgatcaaattt

SEQ ID NO: 155 - DNA, 70 bp, Artificial Sequence
  Description of Artificial Sequence Synthetic probe
cactgctttgagcctcctcatccgagccgaactaggtcagcccggtactttactaggtgacgatcaaatt

SEQ ID NO: 156 - DNA, 70 bp, Artificial Sequence
  Description of Artificial Sequence Synthetic probe
cactgccttgagcctcctcatccgagccgaactaggtcagcccggtactttactaggtgacgatcaaatt

SEQ ID NO: 157 - DNA, 70 bp, Artificial Sequence
  Description of Artificial Sequence Synthetic probe
cactgccttgagcctcctcatccgagccgaactaggtcagcccggtactttactaggtgatgatcaaatt

SEQ ID NO: 158 - DNA, 70 bp, Artificial Sequence
  Description of Artificial Sequence Synthetic probe
cactgccttgagcctcctcatccgagccgaactaggtcagcccggtactttactaggcgacgatcaaatt

SEQ ID NO: 159 - DNA, 70 bp, Artificial Sequence
  Description of Artificial Sequence Synthetic probe
cactgccttgagcctcctcatccgagccgagctaggtcagcccggtactttactaggcgacgaccaaatt

SEQ ID NO: 160 - DNA, 70 bp, Artificial Sequence
  Description of Artificial Sequence Synthetic probe
ctgctttgagcctcctcatccgagccgaactaggtcagcccggtactttactaggtgacgatcaaattta

SEQ ID NO: 161 - DNA, 70 bp, Artificial Sequence
  Description of Artificial Sequence Synthetic probe
ctgccttgagcctcctcatccgagccgaactaggtcagcccggtactttactaggtgacgatcaaattta

SEQ ID NO: 162 - DNA, 70 bp, Artificial Sequence
  Description of Artificial Sequence Synthetic probe
ctgccttgagcctcctcatccgagccgaactaggtcagcccggtactttactaggtgatgatcaaattta

SEQ ID NO: 163 - DNA, 70 bp, Artificial Sequence
  Description of Artificial Sequence Synthetic probe
ctgccttgagcctcctcatccgagccgaactaggtcagcccggtactttactaggcgacgatcaaattta

SEQ ID NO: 164 - DNA, 70 bp, Artificial Sequence
  Description of Artificial Sequence Synthetic probe
ctgccttgagcctcctcatccgagccgagctaggtcagcccggtactttactaggcgacgaccaaattta

SEQ ID NO: 165 - DNA, 70 bp, Artificial Sequence
  Description of Artificial Sequence Synthetic probe
actgctttgagcctcctcatccgagccgaactaggtcagcccggtactttactaggtgacgatcaaattt

SEQ ID NO: 166 - DNA, 70 bp, Artificial Sequence
  Description of Artificial Sequence Synthetic probe
actgccttgagcctcctcatccgagccgaactaggtcagcccggtactttactaggtgacgatcaaattt

SEQ ID NO: 167 - DNA, 70 bp, Artificial Sequence
  Description of Artificial Sequence Synthetic probe
actgccttgagcctcctcatccgagccgaactaggtcagcccggtactttactaggtgatgatcaaattt

SEQ ID NO: 168 - DNA, 70 bp, Artificial Sequence
  Description of Artificial Sequence Synthetic probe
actgccttgagcctcctcatccgagccgaactaggtcagcccggtactttactaggcgacgatcaaattt

SEQ ID NO: 169 - DNA, 70 bp, Artificial Sequence
  Description of Artificial Sequence Synthetic probe
actgccttgagcctcctcatccgagccgagctaggtcagcccggtactttactaggcgacgaccaaattt

SEQ ID NO: 170 - DNA, 60 bp, Artificial Sequence
  Description of Artificial Sequence Synthetic probe
caatagtagaagcaggagcaggaacaggatgaacagtctacccacctctagccggaaatc

SEQ ID NO: 171 - DNA, 60 bp, Artificial Sequence
  Description of Artificial Sequence Synthetic probe
tagtagaagcaggagcaggaacaggatgaacagtctacccacctctagccggaaatctag

SEQ ID NO: 172 - DNA, 60 bp, Artificial Sequence
  Description of Artificial Sequence Synthetic probe
tagtagaagcaggagcaggaacaggatgaacagtctacccacctctagccggaaatccag

SEQ ID NO: 173 - DNA, 60 bp, Artificial Sequence
  Description of Artificial Sequence Synthetic probe
agtagaagcaggagcaggaacaggatgaacagtctacccacctctagccggaaatctagc

SEQ ID NO: 174 - DNA, 60 bp, Artificial Sequence
  Description of Artificial Sequence Synthetic probe
agtagaagcaggagcaggaacaggatgaacagtctacccacctctagccggaaatccagt

SEQ ID NO: 175 - DNA, 60 bp, Artificial Sequence
  Description of Artificial Sequence Synthetic probe
gtctacccacctctagccggaaatctagcccatgcaggagcatcagtagacctaacaatt

SEQ ID NO: 176 - DNA, 60 bp, Artificial Sequence
  Description of Artificial Sequence Synthetic probe
gtctacccacctctagccggaaatccagtccatgcaggagcatcagtagacctaacaatt

SEQ ID NO: 177 - DNA, 60 bp, Artificial Sequence
  Description of Artificial Sequence Synthetic probe
gtctacccacctctagccggaaatctagcccatgcaggagcatcagtagatctaacaatt

SEQ ID NO: 178 - DNA, 60 bp, Artificial Sequence
  Description of Artificial Sequence Synthetic probe
cccacctctagccggaaatctagcccatgcaggagcatcagtagacctaacaattttctc

SEQ ID NO: 179 - DNA, 60 bp, Artificial Sequence
  Description of Artificial Sequence Synthetic probe
cccacctctagccggaaatccagtccatgcaggagcatcagtagacctaacaattttctc

SEQ ID NO: 180 - DNA, 60 bp, Artificial Sequence
  Description of Artificial Sequence Synthetic probe
cccacctctagccggaaatctagcccatgcaggagcatcagtagatctaacaattttctc

SEQ ID NO: 181 - DNA, 60 bp, Artificial Sequence
  Description of Artificial Sequence Synthetic probe
ccacctctagccggaaatctagcccatgcaggagcatcagtagacctaacaattttctcc

SEQ ID NO: 182 - DNA, 60 bp, Artificial Sequence
  Description of Artificial Sequence Synthetic probe
ccacctctagccggaaatccagtccatgcaggagcatcagtagacctaacaattttctcc

SEQ ID NO: 183 - DNA, 60 bp, Artificial Sequence
  Description of Artificial Sequence Synthetic probe
ccacctctagccggaaatctagcccatgcaggagcatcagtagatctaacaattttctcc

SEQ ID NO: 184 - DNA, 60 bp, Artificial Sequence
  Description of Artificial Sequence Synthetic probe
aggatgaacagtctacccacctctagccggaaatctagcccatgcaggagcatcagtaga

SEQ ID NO: 185 - DNA, 60 bp, Artificial Sequence
  Description of Artificial Sequence Synthetic probe
aggatgaacagtctacccacctctagccggaaatccagtccatgcaggagcatcagtaga

SEQ ID NO: 186 - DNA, 60 bp, Artificial Sequence
  Description of Artificial Sequence Synthetic probe
aggatgaaccgtatatccacctttagccggaaatttagcccacgccggagcatcagtgga

SEQ ID NO: 187 - DNA, 60 bp, Artificial Sequence
  Description of Artificial Sequence Synthetic probe
agtctacccacctctagccggaaatctagcccatgcaggagcatcagtagacctaacaat

SEQ ID NO: 188 - DNA, 60 bp, Artificial Sequence
  Description of Artificial Sequence Synthetic probe
agtctacccacctctagccggaaatccagtccatgcaggagcatcagtagacctaacaat

SEQ ID NO: 189 - DNA, 60 bp, Artificial Sequence
  Description of Artificial Sequence Synthetic probe
agtctacccacctctagccggaaatctagcccatgcaggagcatcagtagatctaacaat

SEQ ID NO: 190 - DNA, 60 bp, Artificial Sequence
  Description of Artificial Sequence Synthetic probe
atgaacagtctacccacctctagccggaaatctagcccatgcaggagcatcagtagacct

SEQ ID NO: 191 - DNA, 60 bp, Artificial Sequence
  Description of Artificial Sequence Synthetic probe
atgaacagtctacccacctctagccggaaatccagtccatgcaggagcatcagtagacct

SEQ ID NO: 192 - DNA, 60 bp, Artificial Sequence
  Description of Artificial Sequence Synthetic probe
atgaacagtctacccacctctagccggaaatctagcccatgcaggagcatcagtagatct

SEQ ID NO: 193 - DNA, 60 bp, Artificial Sequence
  Description of Artificial Sequence Synthetic probe
tgaacagtctacccacctctagccggaaatctagcccatgcaggagcatcagtagaccta

SEQ ID NO: 194 - DNA, 60 bp, Artificial Sequence
  Description of Artificial Sequence Synthetic probe
tgaacagtctacccacctctagccggaaatccagtccatgcaggagcatcagtagaccta

SEQ ID NO: 195 - DNA, 60 bp, Artificial Sequence
  Description of Artificial Sequence Synthetic probe
tgaacagtctacccacctctagccggaaatctagcccatgcaggagcatcagtagatcta

SEQ ID NO: 196 - DNA, 60 bp, Artificial Sequence
  Description of Artificial Sequence Synthetic probe
gaacagtctacccacctctagccggaaatctagcccatgcaggagcatcagtagacctaa

SEQ ID NO: 197 - DNA, 60 bp, Artificial Sequence
  Description of Artificial Sequence Synthetic probe
gaacagtctacccacctctagccggaaatccagtccatgcaggagcatcagtagacctaa

SEQ ID NO: 198 - DNA, 60 bp, Artificial Sequence
  Description of Artificial Sequence Synthetic probe
gaacagtctacccacctctagccggaaatctagcccatgcaggagcatcagtagatctaa

SEQ ID NO: 199 - DNA, 60 bp, Artificial Sequence
  Description of Artificial Sequence Synthetic probe
aacagtctacccacctctagccggaaatctagcccatgcaggagcatcagtagacctaac

SEQ ID NO: 200 - DNA, 60 bp, Artificial Sequence
  Description of Artificial Sequence Synthetic probe
aacagtctacccacctctagccggaaatccagtccatgcaggagcatcagtagacctaac

SEQ ID NO: 201 - DNA, 60 bp, Artificial Sequence
  Description of Artificial Sequence Synthetic probe
aacagtctacccacctctagccggaaatctagcccatgcaggagcatcagtagatctaac

SEQ ID NO: 202 - DNA, 60 bp, Artificial Sequence
  Description of Artificial Sequence Synthetic probe
acagtctacccacctctagccggaaatctagcccatgcaggagcatcagtagacctaaca

SEQ ID NO: 203 - DNA, 60 bp, Artificial Sequence
  Description of Artificial Sequence Synthetic probe
acagtctacccacctctagccggaaatccagtccatgcaggagcatcagtagacctaaca

SEQ ID NO: 204 - DNA, 60 bp, Artificial Sequence
  Description of Artificial Sequence Synthetic probe
acagtctacccacctctagccggaaatctagcccatgcaggagcatcagtagatctaaca

SEQ ID NO: 205 - DNA, 60 bp, Artificial Sequence
  Description of Artificial Sequence Synthetic probe
cagtctacccacctctagccggaaatctagcccatgcaggagcatcagtagacctaacaa

SEQ ID NO: 206 - DNA, 60 bp, Artificial Sequence
  Description of Artificial Sequence Synthetic probe
cagtctacccacctctagccggaaatccagtccatgcaggagcatcagtagacctaacaa

SEQ ID NO: 207 - DNA, 60 bp, Artificial Sequence
  Description of Artificial Sequence Synthetic probe
cagtctacccacctctagccggaaatctagcccatgcaggagcatcagtagatctaacaa

SEQ ID NO: 208 - DNA, 70 bp, Artificial Sequence
  Description of Artificial Sequence Synthetic probe
tcaatagtagaagcaggagcaggaacaggatgaacagtctacccacctctagccggaaatctagcccatg

SEQ ID NO: 209 - DNA, 70 bp, Artificial Sequence
  Description of Artificial Sequence Synthetic probe
tcaatagtagaagcaggagcaggaacaggatgaacagtctacccacctctagccggaaatccagtccatg

SEQ ID NO: 210 - DNA, 70 bp, Artificial Sequence
  Description of Artificial Sequence Synthetic probe
caatagtagaagcaggagcaggaacaggatgaacagtctacccacctctagccggaaatctagcccatgc

SEQ ID NO: 211 - DNA, 70 bp, Artificial Sequence
  Description of Artificial Sequence Synthetic probe
caatagtagaagcaggagcaggaacaggatgaacagtctacccacctctagccggaaatccagtccatgc

SEQ ID NO: 212 - DNA, 70 bp, Artificial Sequence
  Description of Artificial Sequence Synthetic probe
gtctacccacctctagccggaaatctagcccatgcaggagcatcagtagacctaacaattttctcccttc

SEQ ID NO: 213 - DNA, 70 bp, Artificial Sequence
  Description of Artificial Sequence Synthetic probe
gtctacccacctctagccggaaatctagcccatgcaggagcatcagtagacctaacaattttctccctcc

SEQ ID NO: 214 - DNA, 70 bp, Artificial Sequence
  Description of Artificial Sequence Synthetic probe
gtctacccacctctagccggaaatctagcccatgcaggagcatcagtagatctaacaattttctcccttc

SEQ ID NO: 215 - DNA, 70 bp, Artificial Sequence
  Description of Artificial Sequence Synthetic probe
gtctacccacctctagccggaaatccagtccatgcaggagcatcagtagacctaacaattttctcccttc

SEQ ID NO: 216 - DNA, 70 bp, Artificial Sequence
  Description of Artificial Sequence Synthetic probe
caggatgaacagtctacccacctctagccggaaatctagcccatgcaggagcatcagtagacctaacaat

SEQ ID NO: 217 - DNA, 70 bp, Artificial Sequence
  Description of Artificial Sequence Synthetic probe
caggatgaacagtctacccacctctagccggaaatctagcccatgcaggagcatcagtagatctaacaat

SEQ ID NO: 218 - DNA, 70 bp, Artificial Sequence
  Description of Artificial Sequence Synthetic probe
caggatgaacagtctacccacctctagccggaaatccagtccatgcaggagcatcagtagacctaacaat

SEQ ID NO: 219 - DNA, 60 bp, Artificial Sequence
  Description of Artificial Sequence Synthetic probe
gagcttctgactcctccctccatcctttctactcttactcgcctcatctatggtagaagc

SEQ ID NO: 220 - DNA, 60 bp, Artificial Sequence
  Description of Artificial Sequence Synthetic probe
gagcttctgactcctccccccatcctttctactcttactcgcctcatctatggtagaagc

SEQ ID NO: 221 - DNA, 60 bp, Artificial Sequence
  Description of Artificial Sequence Synthetic probe
gagcttctgactcctccctccatcctttctactcctactcgcctcatctatggtagaagc

SEQ ID NO: 222 - DNA, 60 bp, Artificial Sequence
  Description of Artificial Sequence Synthetic probe
ctccctccatcctttctactcttactcgcctcatctatggtagaagccggagcaggaact

SEQ ID NO: 223 - DNA, 60 bp, Artificial Sequence
  Description of Artificial Sequence Synthetic probe
ctccccccatcctttctactcttactcgcctcatctatggtagaagccggagcaggaact

SEQ ID NO: 224 - DNA, 60 bp, Artificial Sequence
  Description of Artificial Sequence Synthetic probe
ctccctccatcctttctactcctactcgcctcatctatggtagaagccggagcaggaact

SEQ ID NO: 225 - DNA, 60 bp, Artificial Sequence
  Description of Artificial Sequence Synthetic probe
tcctccctccatcctttctactcttactcgcctcatctatggtagaagccggagcaggaa

SEQ ID NO: 226 - DNA, 60 bp, Artificial Sequence
  Description of Artificial Sequence Synthetic probe
tcctccccccatcctttctactcttactcgcctcatctatggtagaagccggagcaggaa

SEQ ID NO: 227 - DNA, 60 bp, Artificial Sequence
  Description of Artificial Sequence Synthetic probe
tcctccctccatcctttctactcctactcgcctcatctatggtagaagccggagcaggaa

SEQ ID NO: 228 - DNA, 60 bp, Artificial Sequence
  Description of Artificial Sequence Synthetic probe
tgactcctccctccatcctttctactcttactcgcctcatctatggtagaagccggagca

SEQ ID NO: 229 - DNA, 60 bp, Artificial Sequence
  Description of Artificial Sequence Synthetic probe
tgactcctccccccatcctttctactcttactcgcctcatctatggtagaagccggagca

SEQ ID NO: 230 - DNA, 60 bp, Artificial Sequence
  Description of Artificial Sequence Synthetic probe
tgactcctccctccatcctttctactcctactcgcctcatctatggtagaagccggagca

SEQ ID NO: 231 - DNA, 60 bp, Artificial Sequence
  Description of Artificial Sequence Synthetic probe
gctcctgacatagcatttccccgaataaacaacatgagcttctgactcctccctccatcc

SEQ ID NO: 232 - DNA, 60 bp, Artificial Sequence
  Description of Artificial Sequence Synthetic probe
gctcctgacatagcatttccccgaataaacaacatgagcttctgactcctccccccatcc

SEQ ID NO: 233 - DNA, 70 bp, Artificial Sequence
  Description of Artificial Sequence Synthetic probe
atgagcttctgactcctccctccatcctttctactcttactcgcctcatctatggtagaagccggagcag

SEQ ID NO: 234 - DNA, 70 bp, Artificial Sequence
  Description of Artificial Sequence Synthetic probe
atgagcttctgactcccccccccatcctttctactcttactcgcctcatctatggtagaagccggagcag

SEQ ID NO: 235 - DNA, 70 bp, Artificial Sequence
  Description of Artificial Sequence Synthetic probe
atgagcttctgactcctccctccatcctttctactcctactcgcctcatctatggtagaagccggagcag

SEQ ID NO: 236 - DNA, 70 bp, Artificial Sequence
  Description of Artificial Sequence Synthetic probe
agcttctgactcctccctccatcctttctactcttactcgcctcatctatggtagaagccggagcaggaa

SEQ ID NO: 237 - DNA, 70 bp, Artificial Sequence
  Description of Artificial Sequence Synthetic probe
agcttctgactcccccccccatcctttctactcttactcgcctcatctatggtagaagccggagcaggaa

SEQ ID NO: 238 - DNA, 70 bp, Artificial Sequence
  Description of Artificial Sequence Synthetic probe
agcttctgactcctccctccatcctttctactcctactcgcctcatctatggtagaagccggagcaggaa

SEQ ID NO: 239 - DNA, 70 bp, Artificial Sequence
  Description of Artificial Sequence Synthetic probe
cttctgactcctccctccatcctttctactcttactcgcctcatctatggtagaagccggagcaggaact

SEQ ID NO: 240 - DNA, 70 bp, Artificial Sequence
  Description of Artificial Sequence Synthetic probe
cttctgactcccccccccatcctttctactcttactcgcctcatctatggtagaagccggagcaggaact

SEQ ID NO: 241 - DNA, 70 bp, Artificial Sequence
  Description of Artificial Sequence Synthetic probe
cttctgactcctccctccatcctttctactcctactcgcctcatctatggtagaagccggagcaggaact